(19) United States
(12) Patent Application Publication
(10) Pub. No.: US 2010/0004536 A1
(43) Pub. Date: Jan. 7, 2010

(54) METHOD AND APPARATUS FOR ULTRASOUND TISSUE TREATMENT

(76) Inventor: Avner Rosenberg, Beit-Shearim (IL)

Correspondence Address: SMITH FROHWEIN TEMPEL GREENLEE BLAHA, LLC, Two Ravinia Drive, Suite 700, ATLANTA, GA, 30346, US

(21) Appl. No.: 12/167,422

(22) Filed: Jul. 3, 2008

(51) Int. Cl. A61B 8/00; A61N 7/00

(52) U.S. Cl. 600/439

(57) ABSTRACT

Disclosed is a method of treatment of soft tissue where at least two ultrasound transducers are applied to a tissue protrusion and operated to emit the protrusion ultrasound radiation in pulse or continuous operation mode. At least one of the transducers receives a portion of the ultrasound power emitted by the other transducer and transmitted through the protrusion. A controller analyzes the received portion of the ultrasound and employs the data for actively monitoring the ultrasound radiation with tissue coupling. Disclosed is also an apparatus enabling implementation of the method.

### CROSS-REFERENCE TO RELATED APPLICATIONS

This is a non-provisional application filed under 35 USC 111 and claiming priority to United States provisional application for patent having been filed on Jul. 26, 2007 and assigned Ser. No. 60/951,966, which application is hereby incorporated by reference.

### BACKGROUND

The present device is in the field of ultrasound tissue treatment and, in particular, adipose tissue treatment for cosmetic and aesthetic purposes.

Reduction of subcutaneous fat layers, or adipose tissue, is an aesthetic treatment for which there is a growing demand. Among the different physical therapies available, the application of ultrasound is emerging as a leading adipose tissue removal and body shaping technology. Methods associated with this technology are based on the delivery of a dose of electromagnetic or ultrasound energy through the skin of a recipient into the subcutaneous adipose tissue to a volume of tissue to be treated. It is believed that U.S. Pat. Nos. 5,143,063, 5,507,790, 6,071,239, 6,113,558, United States Patent Publication No. 2004/0106867, and U.S. patent application Ser. No. 11/335,181 to the assignee of the present application reflect the current state of this technology.

In order to deliver to the recipient the ultrasound dose, a caregiver attaches an applicator including ultrasound transducers to the recipient skin and transmits the desired dose of energy. The caregiver, however, has no indication of the quality of coupling and transmission of the ultrasound through the tissue and has no information on the interaction of the ultrasound energy with the tissue. There is no information on how successful was the treatment in the change of the aggregation state (composition) of the adipose tissue and when the introduction of ultrasound into the target volume may discontinue and treatment of the next volume may begin. Known technologies for treatment control are based on the measurement of ultrasound reflections. From these reflections they derive the information on the interaction of the ultrasound energy with the tissue. However, the reflected energy does not include sufficient information on the processes taking place in the target tissue volume.

In order to increase the ultrasound power concentrated in a target volume a number of transducers are focused on the same volume and operated simultaneously and continuously. The interaction between the power of ultrasound waves emitted by these transducers emitting one in the direction of the others is a phenomenon that sets emitted power limit. Each transducer has an upper limit of the power density, and the power density at the transducer is the sum of the power emitted by the transducer and the power impinged on it from the other transducers.

For faster development of the ultrasound application technology there is a need to resolve these and other problems and enable a better control over the ultrasound treatment process.

### BRIEF SUMMARY

An embodiment of the present invention includes an apparatus for tissue treatment. In such an embodiment, the apparatus is applied to a recipient tissue with the help of an applicator of ultrasound energy. A control unit via a harness governs operation of applicator. The control unit inter alia includes an ultrasound driver for electrically driving the ultrasound transducers located in applicator, and a processor for monitoring and controlling various functions of apparatus. The control unit may have an input device, such as a keypad that allows the caregiver or apparatus operator to input different commands and desired treatment parameters. Such parameters may be frequency of the ultrasound, pulse duration, pulse repetition rate, intensity of the ultrasound energy to be directed to the treated tissue, RF intensity, vacuum time, and vacuum intensity.

### BRIEF DESCRIPTION OF THE SEVERAL VIEWS OF THE DRAWING

The method and apparatus disclosed are herein presented, by way of non-limiting examples only, with reference to the accompanying drawings, wherein like numerals depict the same elements throughout the text of the specifications.

FIG. 1 is a schematic illustration of an apparatus for tissue treatment in accordance with one exemplary embodiment.

FIGS. 2A and 2B are schematic illustrations of some exemplary embodiments of the applicator for tissue treatment.

FIG. 3 is a schematic illustration of a typical transmitted and induced signal monitored at a transducer operating according to an exemplary embodiment of the method.

FIG. 4 is a schematic illustration of an apparatus for tissue treatment in accordance with an additional exemplary embodiment of the method.

FIG. 5 is a schematic illustration of an additional exemplary embodiment of the applicator for tissue treatment.

FIG. 6 is a schematic illustration of an applicator for tissue treatment in accordance with a further exemplary embodiment of the method.

FIG. 7 is a cross section of the applicator for tissue treatment of FIG. 6.

### DETAILED DESCRIPTION OF EMBODIMENTS OF THE INVENTION

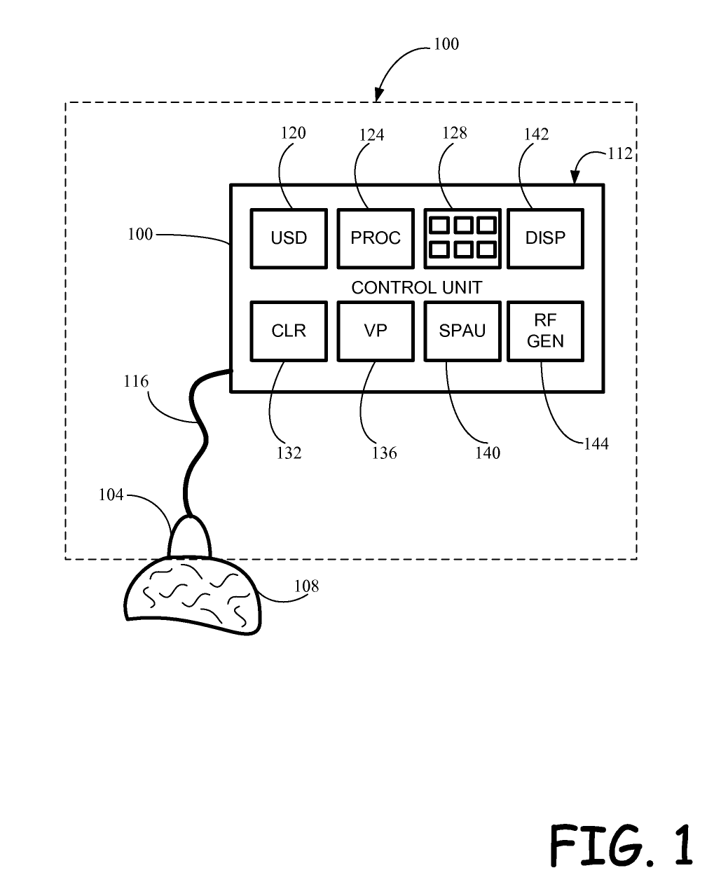

Reference is now made to FIG. 1, which is a schematic illustration of an apparatus for tissue treatment in accordance with an exemplary embodiment. An apparatus 100 applies ultrasound energy to a recipient tissue 108, with the help of an applicator 104. As used herein, the term “tissue treatment” includes skin, dermis, adipose tissue treatment, and such procedures as fat tissue destruction, inducing fat necrosis, inducing fat apoptosis, fat redistribution, adipocyte (fat cell) size reduction, and cellulite treatment. The control unit 112, via a harness 116, governs operation of the applicator 104. The control unit 112, inter alia, includes an ultrasound driver (USD) 120 for electrically driving the ultrasound transducers located in applicator 104, and a processor (PROC) 124 for monitoring and controlling various functions of apparatus 100. The control unit 112 may have an input device, such as a keypad 128 that allows the caregiver or apparatus operator to input to the processor 124 different commands and desired treatment parameters. Such parameters may include, as non-limiting examples, the frequency of the ultrasound, the pulse duration, the pulse repetition rate, the intensity of the ultrasound energy to be directed to the treated tissue, the RF intensity, the vacuum time, and the vacuum intensity.

The control unit 112 may optionally include a cooling means (CLR) 132 for cooling the applicator 104 and the section of skin surface during treatment. The cooling means 132 may be a refrigeration unit that cools a cooling fluid. The cooled fluid flows from the cooling means 132 to the applicator 104 via a tubing, which may be part of the harness 116. A vacuum pump (VP) 136 generates a pressure below atmospheric pressure and causes a region of the tissue to protrude above the surround surface into the inner section of the applicator 104. A vacuum hose embedded in the harness 116 connects the vacuum pump 136 to the applicator 104. A display (DISP) 142 for visual representation of the treatment process parameters may be included in the control unit 112. An optional Radio Frequency (RF) generator (RF GEN) 144 may be located in the control unit 112. The generator 144 provides RF energy to the applicator 104 if needed. Included in the control unit 112 is a signal processing and analyzing unit (SPAU) 140, which may be implemented in hardware and/or software for receiving a feedback from the applicator 104, processing and analyzing the feedback and communicating updated instructions to the applicator 104.

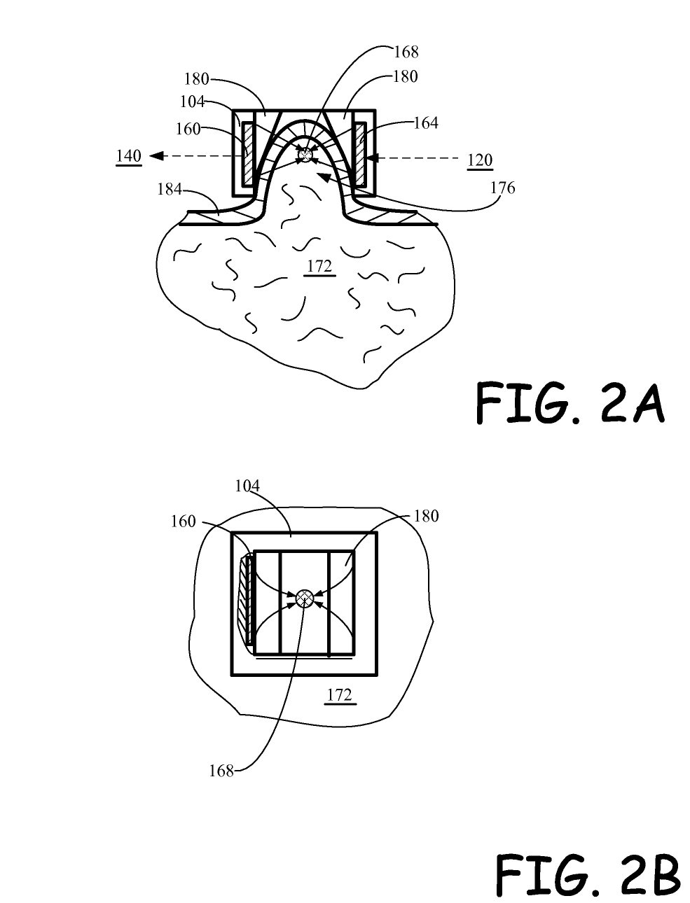

FIGS. 2A and 2B are schematic illustrations of an exemplary embodiment of the applicator for tissue treatment. The applicator 104 contains at least one ultrasound transducer 160 capable of emitting ultrasound power and at least one ultrasound transducer 164, which can receive the ultrasound power or energy emitted from the first ultrasound transducer 160 and transmitted through the tissue protruding into the applicator 104. It is well known in the art that almost all types of ultrasound transducers can be used for both transmitting and receiving ultrasound energy. Accordingly, transducers 160 and 164 can be similar or identical and both of them may operate in either emitting or receiving mode. Ultrasound driver 120 provides electric power to the ultrasound transducers 160 and 164. As used herein, the term “transducer” includes ultrasound emitter and ultrasound receiver.

The applicator 104 is adapted for application to the tissue of recipient 108 in a region of tissue to be treated. The applicator 104 may, in addition to transducers 160 and 164, optionally includes one or more pairs of RF electrodes directing RF current supplied by generator 144 into the treated tissue. (Some of the applicator elements are omitted for clarity of the explanation.) When RF electrodes are included in applicator 104, the processor 124 may monitor the electrical impedance between electrodes and determine the RF coupling quality. The processor 124 may also monitor the effectiveness of the treatment by monitoring variations of temperature in the tissue, since the electrical conductivity of the tissue is temperature dependent.

FIGS. 2A and 2B illustrate ultrasound transducers 160 and 164 as a pair of planar transducers, although it should be clear that this is simply one, non-limiting example of the transducer arrangement. Other non-limiting examples may include curved and/or phase array types. As illustrated in FIG. 7, the curved transducers may have a shape or be arranged such as to encircle the treated tissue. In addition, it should be appreciated that the number of transducers is not limited to two, and additional ultrasound transducers (or receivers) being in contact with the tissue protrusion 176 and emitting ultrasound waves such that they propagate from one transducer to another through the protruded tissue may be added to the applicator.

U.S. application for patent having Ser. No. 11/335,181, inter alia, describes exploiting the high flexibility of tissue, and generating a protrusion 176 out of the body surface. This feature increases the area of the skin/tissue for delivering energy into the target tissue volume and improves the efficiency of the treatment. Negative pressure (vacuum) provided by vacuum pump 136 (FIG. 1) to applicator 104 creates within the inner section of applicator 104, a protrusion 176 of tissue 172. Alternatively, mechanical manipulations of tissue 172 may also be used to create the protrusion. A pair of ultrasound transducers 160 and 164 is attached in proximity to that protrusion and the transducers radiate typically, in a direction parallel to the undisturbed skin surface, or at least as close as possible to that optimal angle. This allows concentrating larger amounts of energy in the target volume 168, while much less radiation arrives at deeper body tissue. Optionally, wedge shaped ultrasound coupling means 180 having acoustical properties similar to those of soft tissues may be used to improve ultrasound to soft tissue coupling efficiency. The upper tissue layers such as skin or dermis 184 comes into contact with the wedge shaped ultrasound coupling means 180 to create a relatively consistent medium for the ultrasound energy to propagate through.

Applicator 104 is configured to deliver ultrasound energy to a region of subcutaneous tissue to be treated, such as to generate a sufficient energy concentration for creating the desired treatment effects. A single transducer can deliver ultra sound energy, however, a limited amount thereof, that is termed herein as the rated amount of ultrasound energy. There is also a limit to the degree of focusing the ultrasound energy and the size of the focusing volume of the ultrasound energy. U.S. application for patent Ser. No. 11/335,181 filed by the assignee of the present application teaches the use of at least a pair of transducers, such as transducers 160 or 164, focused to the same target volume 168 of the treated tissue 172.

Transducers 160 and 164 can be operated simultaneously to deliver energy at the same target tissue volume 168 to increase the amount of energy coupled to the tissue volume 168. In one embodiment, both transducers are emitting ultrasound at the same frequency and with fixed relative phases. In such case, ultrasound velocity fields combine. Ultrasound power density is proportional to the square of absolute value of the sound velocity. Therefore, for two transducers radiating equal power densities one can expect the power density at the target tissue to be up to four times the power emitted by a single transducer. Practically, this theoretical limit cannot be reached since each transducer has a limited power rating. When the ultrasound wave emitted by the first transducer and transmitted through the tissue in protrusion 176 reaches the second transducer, it interferes with the ultrasound wave emitted by the second transducer. The second transducer cannot handle the power density which it emits and the additional power density arriving from the first transducer. As noticed above, each transducer has a limited power rating. This rating reduces the ultrasound power, which can be emitted by the second transducer and vice versa.

According to an exemplary embodiment of the apparatus and method, operation of the transducers in a pulsed mode alleviates this problem. The duration of the pulse should be such that when the ultrasound wave front emitted by the first transducer arrives at the second transducer, the second transducer has already finished emitting and transmitting ultrasound. Pulses and, in particular, short pulses are never ideal square pulses. They generally have a rise time and fall time and a further optimization of the pulse length could be done to obtain maximum power as well as maximum energy per pulse in the tissue. A textbook definition of pulse length or pulse duration is the width between half power points, or what is called the −3 dB points. Accordingly, for the optimal situation it is possible to set the half power (−3 dB) width of the pulse to be equal to, or smaller than, the transition time of the ultrasound from one transducer to another.

The author of the present method has experimentally proved that short pulses are at least as good for tissue treatment as longer ones and produce the same ultrasound energy-tissue interaction results and, in particular, fat tissue cell destruction. In order to compare influence of different pulse durations, the same average ultrasound power ([peak power]×[duty cycle]) was maintained in all of the tests.

An important advantage of using short ultrasound pulses is that the power density at the outer layers 184 is small as compared with power density at the fat tissue. According to one embodiment, two or more ultrasound transducers 160 and 164 emitting radiation of the same frequency from opposite sides of the protrusion into the tissue 172, are focused at the same volume 168. If the pulse (half power width) is shorter than the transition time between the transducers, the ultrasound waves can combine at the focal volume 168 to four times the power of a single transmitting transducer. Thus, high power density exists only at the focal volume 168 of the protrusion 176 where the fat tissue resides. The ultrasound power density at the outer layers 184 is substantially lower than the power density in the focal volume 168, due to the differences in arrival times of the ultrasound pulse. The peak of the power of the pulse arrives at that layer from the nearest transducer before the peak of power arriving from the other transducer. This avoids any potential damage to the outer layers 184.

If the duration of the transmitted pulse is shorter than the time required for the ultrasound emitted by the first transducer to travel through the tissue to the second transducer, then almost all of the pulses emitted and transmitted through the tissue wave arrive at the second transducer after the second transducer has finished emitting ultrasound and may serve as a receiver. If a pulse equals twice the transition time, half of the pulse is received after the end of the transmission. The signal induced in the second transducer may, for example, be voltage induced in one transducer by the ultrasound emitted by the other transducer. The signal might be sampled and analyzed by means well known in the art. Thus, the use of short pulses enables real time diagnostic of the ultrasound transmission through the tissue and analyses of the ultrasound interaction with the treated tissue, based on the same transducers used for the power transmission into the tissue, without the need for additional specific receiver transducers.

A numerical example provides a better illustration of the pulse durations discussed. For example, the distance between the transducers may be 40 to 50 mm. The average sound velocity in the tissue is about 1500 m/sec. The pulse transition time would be about 27 to 33 microseconds. Therefore, if pulse duration is shorter than this time and the pulse is transmitted simultaneously by both transducers when the beginning of the ultrasound wave transmitted by one transducer arrives at the second one, this has already stopped transmitting and can function as a receiver. In practice ultrasound pulses are not ideal rectangular, and have rise and fall time which have to be considered when selecting the optimal pulse length. While the best way is to use a pulse equal to or shorter than the transmission time, one can use a longer pulse and sample only the trailing part of the pulse, which arrives after the end of the transmission.

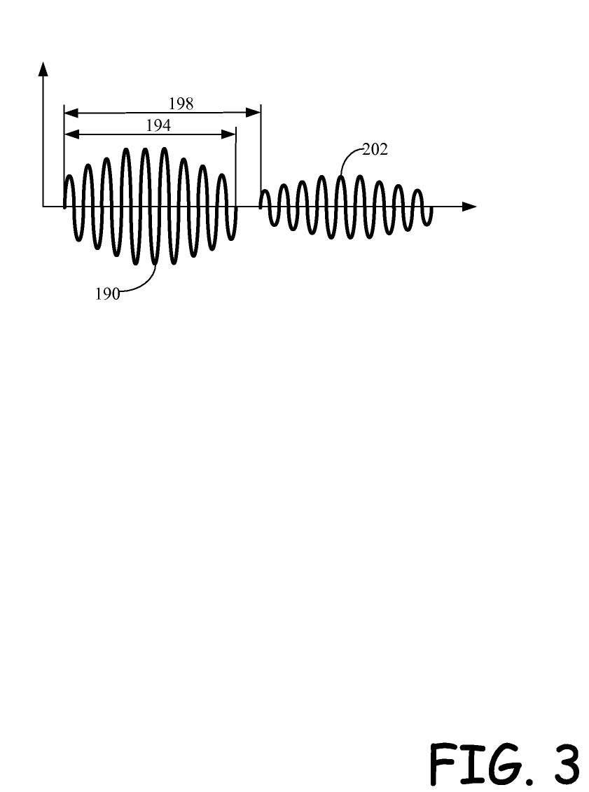

There is no particular need to use identical transducers. The receiving transducer should be capable of receiving at least part of the frequency spectrum transmitted by the transmitting transducer. FIG. 3 shows an example of a typical image of signals at each of the transducers 160 or 164. Numeral 190 marks the transducer driving voltage. It continues for a certain time marked by numeral 194. Upon completion of the transmission time 198 the ultrasound wave from the other transducer arrives, and induces at the transducer signal 202, which is sampled and communicated to the signal processing and analyzing module 140. Typically, the amplitude of the induced signal 202 will be lower than that of the transmitted signal due to ultrasound propagation losses and limited transducer efficiency. Processing of both signals gives valuable data on the transmitted and the received ultrasound. The signal sampling on the transducer can be done by any standard techniques, such as capacitive coupling, magnetic coupling, or resistive coupling.

Ultrasound waves transmitted through the protruding tissue carries with it information on the interaction of the ultrasound with the tissue and, in particular, on the quality of coupling of the ultrasound from the transducers to the tissue and the transmission through the tissue. Analysis of the signals induced at the receiving transducer by the ultrasound pulses emitted by the transmitting transducer may include at least one of the following parameters: the signal transmission time, or what may be termed as the time it takes for the signal emitted by the first transducer to arrive at the second transducer; the peak signal intensity at the front of the received pulse, and intensity fluctuations following the front of the pulse.

The time of arrival is a direct measure or indication of the average sound velocity inside the tissue. The average sound velocity gives indications on the aggregate state of the treated tissue. For example, since the sound velocity on the skin is higher than that in the fat, thicker skin will increase the average sound velocity and reduce the pulse arrival time. Sound velocity in tissues is known to be temperature dependent. Therefore, the measurement of sound velocity can give data on the change of tissue temperature and the efficiency of the treatment associated with it. Monitoring tissue temperature is important for controlling the treatment and avoiding unwanted damage to tissue. Heating of tissue is generated by the ultrasound energy and/or by optional introduction of RF energy, which is applied to the treated tissue as disclosed in the U.S. patent application Ser. No. 11/335,181 assigned to the same assignee as this present invention. Very large variations of ultrasound travel time from a nominal and known value will indicate an undesired path of ultrasound propagation.

The peak intensity at the front of the pulse is a direct indication that the transmitting transducer is working properly and that the coupling of ultrasound to tissue is correct and accordingly the transmission through tissue is good.

Experiments conducted by the author of the disclosed method indicated that when the ultrasound is applied in continuous mode and the threshold of fat destruction is reached, the induced signal intensity begins to fluctuate. When ultrasound is applied to the tissue in a pulse mode, the fluctuations start after the front of the pulse. Therefore, measurement of intensity fluctuations following the received pulse front is an indication of the effectiveness of the treatment. In addition, it was observed that when the fat tissue destruction process was completed, the transmitted signal fluctuations stopped, and transmission through tissue became high and stable. This received pulse behavior provides good real time information at the start and the end of the fat tissue destruction process.

This information, together with additional information extracted from the processing of the induced signal is communicated to processor 124 (FIG. 1) and may provide data to be employed for real time, active monitoring of the ultrasound radiation with tissue coupling process. Corrective actions may include change of operational parameters of the transmitted ultrasound or of the vacuum suction which creates the protrusion (this parameter has an effect on the coupling), or of the RF energy if included in the applicator (RF energy changes the tissue temperature, which can affect the ultrasound propagation and interaction effects in the tissue.) The operational parameters of the transmitted ultrasound are at least: pulse peak power, pulse length, pulse repetition rate, relative phase between transducers and focusing scheme if phased arrays are used. The peak ultrasound intensity may be set to exceed the threshold power for the desired tissue treatment effects, but should not get too high to reduce the risk of collateral damage.

Based on the monitored signal analyses communicated to processor 124 by processing unit 140, processor 124 (FIG. 1) may undertake corrective action and change automatically one or more of the above listed operational parameters. Such apparatus will operate in a closed loop mode and intervention of the caregiver or apparatus operator will be not required. Alternatively, the information extracted from the processing of the induced signal may be displayed on display 142 (FIG. 1) providing the caregiver or apparatus operator with the possibility of manual operation of the apparatus. Manual change of ultrasound operational parameters is performed by entering them via keypad 128.

The real time ultrasound radiation with tissue coupling (or interaction) monitoring process is applicable to long pulse or continuously radiating transducers. In such cases, one transducer emits ultrasound energy required for treatment and the other transducer serves as a receiver only for at least part of the time. The control system 124 can be set for time division of tasks such that for most of the time, both transducers are transmitting, with no induced signal analysis of the transmitted energy. At the time that one of the transducers is not driven to emit ultrasound, it may function as a receiver. The processing and analyses of the induced signal are executed in a similar way. However, part time signal monitoring does not provide information on full power density at the target tissue volume, since one of the transducers is not transmitting at the monitoring time. Application of phased array transducers can alleviate this problem and offer additional advantages for the treatment of tissue.

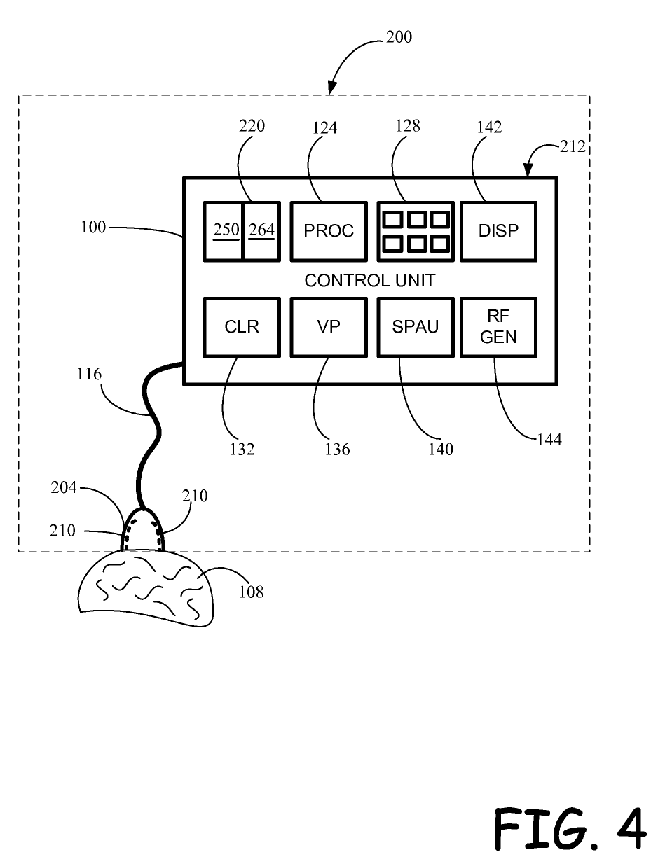

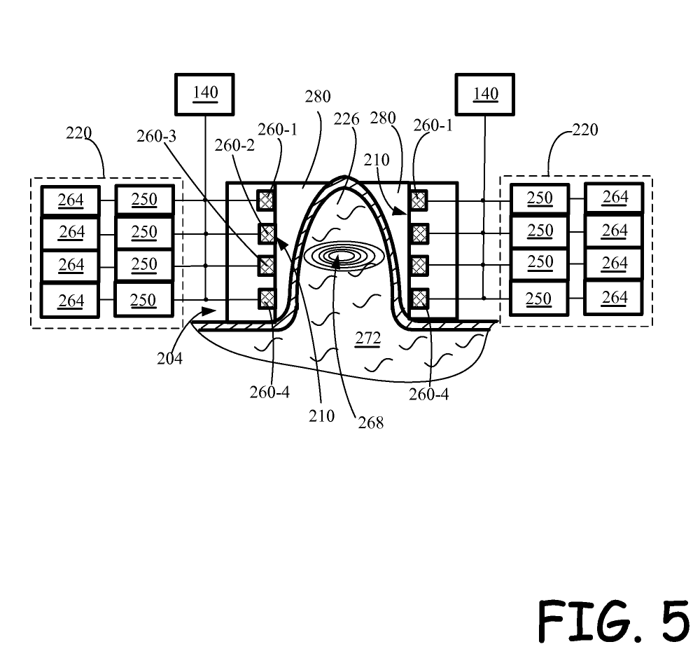

FIG. 4 is a schematic illustration of another embodiment of the apparatus for tissue treatment in accordance with an exemplary embodiment of the method. The apparatus 200 has a number of features distinct from the apparatus 100. The applicator 204 includes phased array ultrasound transducers 210. An independent driver channel 250 located in the drivers unit 220 of the controller 212 drives each individual element 260-1 through 260-4 (FIG. 5) of transducers 210. The phase generators 264 control the phase of each element 260. The control unit 212 controls the phasing program of elements 260. The phasing program is configured to obtain a focal volume 268 (FIG. 5) at the desired location inside tissue 272.

FIG. 5 is a schematic illustration of an additional exemplary embodiment of the applicator for tissue treatment. The applicator 204 includes phased array ultrasound transducers 210 positioned face to face. The transducers 210 deliver ultrasound waves into tissue 272. Optionally, the coupling means 280 may be employed to improve ultrasound to tissue coupling. The tissue protrusion 226 is generated by vacuum suction or mechanical manipulation. Each transducer 210 is built from a plurality of elements 260. Although only four elements are shown in each transducer, their number and size may be chosen according to a known practice of phased array design. Independent driver channel 250 drives each element 260. The phase generators 264 control the phase of each element 260. The control unit 212 controls the phasing program of elements 260, such that they are focused in a target volume 268.

Two elements of each transducer, for example elements 2 and 3, are sampled. Any number of elements can be chosen for sampling and induced signal analysis. The more elements that are selected, the more accurate is the transmission data. Sampling can be done by any known coupling means. The signal produced by the coupling means is fed to the processing and analyzing module 140 and the processor 124. As explained earlier, the processor 124 can automatically change treatment parameters, stop treatment in the case of poor coupling, and/or display the information for a caregiver or operator decision. All the induced signal analyses described above are mutatis mutandis applicable to the phased array transducers.

Furthermore, the application of phased array transducers enables sophisticated analysis of the data received and better treatment control. For example, the operator may instruct the control system to perform a scanning of the focal volume. The control system computes the phases to get the focal volume at the required position. If the ultrasound transmission is sufficient for each focal volume, a specific set of voltages is expected at the sampled elements of the arrays. Any deviation from proper transmission will be noticed and can be analyzed to get data on the transmission. For example, elements 2 and 3 generate equal voltages when the focal point is aimed between them. Occasional shift of the focal volume could be detected by a difference in the voltage generated by elements 2 and 3. Short pulses are used with phased array transducers in a way similar to the uniform phase transducers. Phased arrays enable easier monitoring of the transmission with long pulses or continuous transmission. The control system 240 can select and sample a specific element for a desired time. During that time, the selected element will not be driven for transmission and used as a receiver. The loss of power at the focal tissue volume is small if the number of elements in the array is sufficiently large. For example, in a 32 element array, one element not emitting will reduce the emitted radiation by 1/32 only. A more sophisticated control scheme can sample elements according to a time and position program.

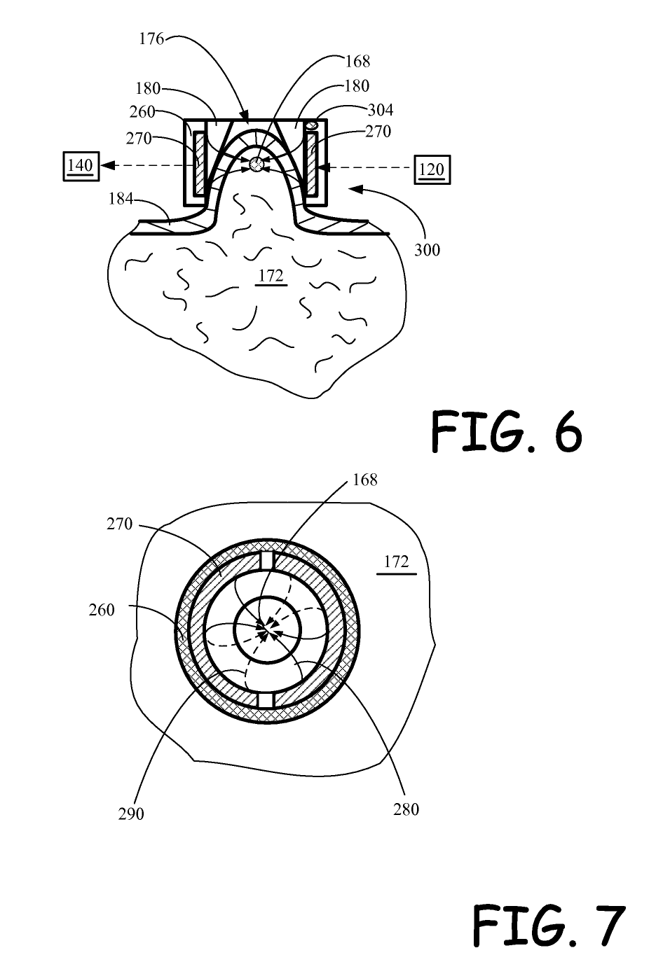

The author of the present method has experimentally found that when the ultrasound pulses break the fat tissue cell, an unarmed ear hears a sound. The signal is a type of “knocking” noise accompanying each ultrasound pulse. FIG. 6 is a schematic illustration of an applicator for tissue treatment in accordance with a further exemplary embodiment of the method. The applicator 300 includes an acoustical microphone 304. The acoustical microphone 304 collects audible sound from the treated tissue volume, and filters only those sounds, which are accompanying or synchronized with an ultrasound pulse. The transducers 270, as shown in FIG. 7 could be curved, regular or phase array transducers that may have a shape or be arranged such as to encircle the treated tissue 172. Different operating sections of phase array transducers 270 shown in FIG. 7, which is a cross section of the applicator for tissue treatment of FIG. 6, may be operated such as to concentrate the ultrasonic field in a common target focal volume 168. Numerals 280 (solid lines) and numeral 290 (phantom lines) show an ultrasonic field produced by transducers 270 and directed from different directions to be concentrated in focal volume 168.

The method and apparatus disclosed give the caregiver a clear indication of the quality of coupling and transmission of the ultrasound through the tissue, provide information on the interaction of the ultrasound energy with the tissue, and improve the quality of the treatment.

Induced signal power fluctuations provide information regarding how successful the treatment was in the change of the aggregation state of the adipose tissue and when the introduction of ultrasound into the target volume may be discontinued and the treatment of the next volume may begin.

By using short pulses, high power density can be obtained in the tissue to be treated without exceeding the power rating of the transducers.

Another benefit of the short pulse is that the ultrasound power density at the outer tissue layers is substantially lower than the power density in the focal volume. This avoids any potential damage to the outer tissue layers.

Short pulses enable transmitting and receiving of the same by a transducer, and separating the transmitted from received signals by their propagation time delay.

The application of phased arrays enables time-sharing between transmission and receiving tasks with negligible loss of power.

Occasional shift of the focal volume or other disturbances of ultrasound propagation could be detected by difference in the voltage generated by receiving elements of a phased array transducer.

While the exemplary embodiment of the present method and apparatus has been illustrated and described, it will be appreciated that various changes can be made therein without affecting the spirit and scope of the apparatus and method. The scope of the method, therefore, is defined by reference to the following claims.

What is claimed is:

1. A method of treatment of soft tissue, said method comprising:
a) placing at least two ultrasound transducers in proximity to a tissue protrusion;
b) emitting ultrasound energy into the tissue protrusion in a pulse or continuous operation mode from said at least two ultrasound transducers;
c) receiving by at least one of said transducers at least a portion of the ultrasound energy emitted and transmitted through said tissue protrusion by the other transducers; and,
d) analyzing said received portion of the ultrasound energy to provide data for real time monitoring of at least one of: a coupling of the ultrasound energy to the tissue protrusion, an aggregate state of said tissue volume, a sound velocity in said tissue, and a beginning of fat tissue destruction.

2. The method according to claim 1, wherein in said pulse operation mode, the pulse duration of the ultrasound energy eliminates crosstalk between said transducers.

3. The method according to claim 1, further comprising operating said transducer in the pulse operation mode and selecting a duration of said pulse, such that half power width of said pulse is shorter than the time said ultrasound energy emitted by one of said transducers reaches the second transducer.

4. The method according to claim 1, wherein each of said transducers emits into a common target tissue volume located in said tissue protrusion.

5. The method according to claim 1, further comprising the step of applying a vacuum to create the tissue protrusion, wherein said tissue protrusion increases the area of the tissue through which ultrasound energy is delivered into the target tissue volume.

6. The method according to claim 1, wherein the step of emitting ultrasound energy further comprises radiating ultrasound energy in a direction parallel to the skin surface that is not part of the tissue protrusion.

7. The method according to claim 1, wherein at least one of said transducers upon completion of the step of emitting ultrasound energy, operate in an ultrasound radiation receiving mode.

8. The method according to claim 1, wherein in pulse operation mode, the step of analyzing the received portion of the ultrasound energy further comprises analyzing parameters of said received ultrasound energy pulsed signal including at least one of the signal transition time, peak received intensity at the front of the pulse, intensity fluctuation following the front of the pulse, and phase shift.

9. The method according to claim 1, wherein in continuous operation mode, the step of analyzing the received portion of the ultrasound energy further comprises analyzing parameters of said received portion including at least one of the peak signal intensity, intensity fluctuations, and phase shift.

10. The method according to claim 1, further comprising the step of adjusting of operational settings of the ultrasound transducers based on the results of said analyzing step.

11. The method according to claim 1, wherein said step of analyzing further comprises determining state of fat tissue destruction, wherein fluctuations of said received signal power indicate destruction of said fat tissue and the absence of fluctuations of said received signal power indicate completion of the fat tissue destruction process.

12. The method according to claim 1, wherein said step of analyzing further comprises determining the effectiveness of the treatment, wherein the intensity of said received at least a portion of the ultrasound energy is an indication on the effectiveness of the treatment.

13. The method according to claim 1, wherein an audible signal indicates the fat tissue cells break and further comprising the step of detecting such audible signal.

14. The method according to claim 1, further comprising the step of applying an RF power concurrently with said ultrasound energy.

15. A method of treatment of soft tissue, said method comprising:
a) placing proximate to a tissue protrusion at least two ultrasound transducers;
b) causing said ultrasound transducers to emit ultrasound energy into a common tissue volume in said protrusion and operating said transducers at least part of the time to emit ultrasound energy and at least part of the time to receive the emitted ultrasound energy;
c) receiving the ultrasound energy transmitted through said protrusion; and
d) processing the received ultrasound energy by employing it for actively monitoring said ultrasound energy with tissue coupling.

16. The method according to claim 15, wherein the step of causing said ultrasound transducers to emit further comprises causing said ultrasound transducers to emit ultrasound energy in either a continuous or a pulse mode.

17. The method according to claim 15, wherein said step of processing of the received ultrasound energy enables setting operational parameters of said ultrasound transducers and where the setting is performed in one of automatic or manual mode.

18. The method according to claim 15, further comprising the step of identifying fat tissue destruction, wherein intensity fluctuations of said received ultrasound energy indicate the threshold of said fat tissue destruction.

19. The method according to claims 15, wherein said step of processing the received ultrasound energy further comprises analyzing operational parameters of said ultrasound energy including at least one of the pulse peak power, pulse length, pulse repetition rate, relative phase between transducers and focusing scheme.

20. The method according to claims 18, wherein said step of processing the received ultrasound energy further comprises analyzing operational parameters of said ultrasound energy including at least one of the pulse peak power, pulse length, pulse repetition rate, relative phase between transducers and focusing scheme.

21. The method according to claim 15, wherein an audible signal detectable by unarmed ears indicates said fat tissue cells break and further comprising the step of detecting the audible signal.

22. A method of operating an applicator used for treatment of soft tissue, said method comprising:
a) providing an applicator with at least two ultrasound emitting transducers, and
b) operating said transducers in a pulse mode, such that said pulse duration of an ultrasound energy signal is shorter than the time it takes for the ultrasound energy signal front emitted by one transducer to propagate through soft tissue under treatment and arrive at the second transducer.

23. The method according to claim 22, wherein the step of operating said transducers further comprises applying the ultrasound energy signal to a skin protrusion formed by said applicator.

24. The method according to claim 22, further comprising the step of applying an RF energy signal to the protrusion in at least overlapping with ultrasound periods.

25. A method of avoiding damage to soft tissue during ultrasound tissue treatment, said method comprising:
a) forming a soft tissue protrusion;
b) placing at least two transducers proximate to the soft tissue protrusion;
c) emitting an ultrasound energy signal by said transducers into a common target volume in said soft tissue protrusion;
d) operating said transducers to deliver the ultrasound energy signal in pulses to said target volume, such that high ultrasound radiation density is generated in said target volume; and
e) arranging the operational periods of said transducers such that ultrasound energy signal emitted by one transducer only passes at a time through a section of outer tissue.

26. An apparatus for ultrasound treatment of soft tissue, said apparatus comprising:
a) an applicator for generating a protrusion of said soft tissue, said applicator having at least one ultrasound energy transmitting transducer and at least one ultrasound energy receiving transducer; and
b) a controller for driving said applicator and having a signal processing and analyzing unit for processing an ultrasound energy signal transmitted by said transmitting transducer and received by said receiving transducer.

27. The apparatus according to claim 26, wherein the applicator generates the tissue protrusion by vacuum or mechanical means.

28. The apparatus according to claim 26, wherein said signal processing and analyzing unit is operable to detect and analyze ultrasound energy signal parameters including at lest one of the parameters including the signal transition time, the peak received intensity at the front of the pulse, the intensity fluctuation following the front of the pulse, and the phase shift.

29. The apparatus according to claim 26, wherein said transducer is selected from a group of transducers including a uniform phase transducer, a curved transducer, or a phased array transducer and wherein at least a section of said phased array transducer operates as a receiver.

30. The apparatus according to claims 26, wherein the ultrasound energy receiving transducer provides a feedback on said treatment process to said controller and wherein based on said feedback the controller sets at least one of the operating parameters for the transmitting transducer.

31. The apparatus according to claims 29, wherein the ultrasound energy receiving transducer provides a feedback on said treatment process to said controller and wherein based on said feedback the controller sets at least one of the operating parameters for the transmitting transducer.

32. The apparatus according to claims 26, wherein said signal processing and analyzing unit is operable to detect and analyze ultrasound energy signal parameters including at lest one of the pulse peak power, the pulse length, the pulse repetition rate, the relative phase between transducers and the focusing scheme.

33. The apparatus according to claims 29, wherein said signal processing and analyzing unit is operable to detect and analyze ultrasound energy signal parameters including at lest one of the pulse peak power, the pulse length, the pulse repetition rate, the relative phase between transducers and the focusing scheme.

34. The apparatus according to claim 26, wherein said applicator includes a microphone for sensing ultrasound to tissue coupling effects producing an audible signal and where said microphone communicates with said control unit.

35. The apparatus according to claims 34, wherein the controller, based on the microphone signal, sets operational parameters of said ultrasound.

36. The apparatus according to claim 26, wherein said applicator further comprises at least a pair of RF electrodes.

37. An apparatus for ultrasound treatment of soft tissue, said apparatus comprising:
a) an applicator for forming a soft tissue protrusion, said applicator comprising:
i) at least one ultrasound transducer applying ultrasound radiation to said soft tissue protrusion, and
ii) at least one ultrasound radiation receiver for receiving the ultrasound radiation transmitted through said soft tissue protrusion; and
b) a controller for controlling operation of said applicator, said controller comprising:
i) an ultrasound driver for driving said ultrasound transducer; and
ii) a processing and analyzing unit for processing the signal received by said ultrasound radiation receiver and setting operation parameters of said ultrasound driver.

38. The apparatus according to claim 37, wherein said applicator further comprises a pair of RF electrodes and a microphone.

39. The apparatus according to claim 37, wherein said controller further comprises an RF generator communicating with said RF electrodes.

40. An apparatus for ultrasound treatment of soft tissue, said apparatus comprising:
a) an applicator for forming a soft tissue protrusion, said applicator comprising:
i) at least two ultrasound transducers applying ultrasound radiation to said soft tissue protrusion, wherein at least a section of each of said transducers operates as a receiver;
ii) a pair of RF electrodes, and
iii) a microphone for monitoring audible effects of ultrasound to tissue coupling, and
b) a controller for controlling operation of said applicator.

41. The applicator according to claim 40, further comprising:
a) connection to a source of vacuum for forming said soft tissue protrusion; and
b) a connection to a signal processing and analyzing unit for processing a received ultrasound signal by the receiver ultrasound signal.

42. A method for ultrasound radiation treatment of soft tissue, said method comprising the steps of:
a) creating a tissue protrusion in the soft tissue;
b) placing at least two ultrasound transducers in proximity to the tissue protrusion and on substantially opposing sides of the tissue protrusion;
c) emitting an ultrasound energy signal into the tissue protrusion from said at least two ultrasound transducers;
d) stopping the emission of the ultrasound energy signal at a time less than the time required for the ultrasound energy signal to propagate through the issue protrusion;
e) receiving by at least one of said transducers at least a portion of the ultrasound energy signal emitted and transmitted through said tissue protrusion by the other transducers; and,
d) analyzing said received portion of the ultrasound energy ascertain characteristics of the tissue protrusion.
